(19) United States
(12) Patent Application Publication
(10) Pub. No.: US 2026/0165300 A1
(43) Pub. Date: Jun. 18, 2026

(54) Methods And Apparatus For Generating And Using A Simulated 3D Model of an Animal's Face for providing identification and clinical information

(71) Applicant: Scott Pless, Las Vegas, NV (US)

(72) Inventor: Scott Pless, Las Vegas, NV (US)

(21) Appl. No.: 18/979,032

(22) Filed: Dec. 12, 2024

(51) Int. Cl. A01K 29/00; G01S 17/894; G06T 7/00; G06T 17/00; G06V 20/64; G06V 40/10; G16H 10/60; G16H 50/50

(52) U.S. Cl. CPC A01K 29/005; G06T 7/0014; G06T 17/00; G06V 20/647; G06V 40/10; G16H 10/60; G16H 50/50; G01S 17/894; G06T 2207/30004; G06T 2210/41

(57) ABSTRACT

Methods for identification of animals and providing clinical information using facial scanning and 3D hologram generation along with LiDAR data, are provided for veterinary, animal welfare, livestock, and other applications that quickly identify particular animals and clinical information without using invasive techniques and needed interventions, as well as improving animal-related tracking and record keeping.

### FIELD OF THE INVENTION

The invention relates to methods for the identification of individual animals and for clinical uses, such as the identification of any clinical records for the animal, using a simulated 3D model of the animal's face.

### BACKGROUND OF THE INVENTION

Current methods of identification of individual animals and certain clinical applications have several drawbacks. Intrusive ear or other tags that are used on livestock (e.g., cattle, pigs, chickens) have many disadvantages. They can cause pain when applied by making a hole in an ear or from using other attachment means, for example, and they are sometimes unintentionally pulled off causing additional pain and possible infection, also thereby losing the identification and related clinical information for the animal. Collars and microchips are used on pets, which are also often intrusive, and which can get caught on protrusions and cause strangulation and/or possible infections when applied. These identification methods are not related to the clinical history of the animals and thus they do not assist in needed interventions and the diagnosis and treatment of disease. There is also currently great interest in tracking livestock and food products to determine their origin and to avoid disease and contamination.

Better methods for identifying individual animals and relevant clinical information, such as possible past disease or injury, are needed to provide improved identification of lost animals, the medical and other history of an animal, the origin and ownership of animals, possible history of disease, a wide variety of other clinical applications, and other reasons that follow when individual animals can be identified and imaging data of an animal's face analyzed.

### SUMMARY OF THE INVENTION

Preferred embodiments of this invention comprise methods and applications for the use of imaging devices (e.g., cameras on mobile phones, smart phones, tablets, and other devices) to video scan the face of an individual animal so that the animal, and any clinically relevant information, such as vaccination status and any disease history, can be identified.

The term “medical abnormality” is used broadly herein to include disease states, injuries, symptoms, diagnostic indications, skin conditions, changes in appearance or demeanor, and/or other similar characteristics of an animal that is not presenting, behaving, or exhibiting normally or otherwise as expected or desired.

The most preferred embodiments use a mobile phone and artificial intelligence to use a video scan of an animal's face, collect and process the scanning data (e.g., hologram data, LiDAR data) to create a 3D model of the animal's face, correlate the 3D model with identification and other information provided by the user, process the data and information with AI applications, and store the data and correlated identification information (e.g., cloud-based storage). When the animal's face is scanned a second time, the scanning data and/or 3D model is compared to the stored scanning data and/or 3D model (e.g., stored in cloud-based storage) to determine the identification of the animal and whether the correlated identification information applies to the animal. In certain of these embodiments, once the animal is identified, the correlated identification information can be updated (e.g., weight gain and other changes and updates).

The correlated identification information can include, or be used to acquire, other specific information concerning the animal, such as medical records, vaccination status, weight records, feed records (e.g., in a dairy or animal milking facility, with production animals), owner contact/origin information, and other information. In certain preferred embodiments, the information collected is analyzed to determine whether the animal is expressing any symptoms of disease or other clinically relevant information and whether particular clinical issues may apply.

In certain of the most preferred embodiments, methods for identification of an animal and providing clinically-relevant information concerning the animal to a user are provided. The methods comprise: (a) collecting video imaging data and LiDAR data of the animal's face. A device with a camera and computing and memory components can be used (e.g., iPhone, smart phone, mobile phone, handheld device, tablet, permanently installed device). Next can be (b) interpolating the video imaging data with artificial intelligence using 3D Gaussian Splatting to create a model of the animal's face. After that, can be (c) assimilating the model of the animal's face with the LiDAR data to create a simulated 3D model of the animal's face, (d) comparing the simulated 3D model of the animal's face with a database of identification and clinical information of animals to locate any identification and clinical information applicable to the animal; and (e) outputting any located identification and clinical information for the animal to the user.

In some embodiments, the (d) comparing comprises application of facial recognition algorithms. In addition, in some embodiments, the (d) comparing further comprises incorporating contextual data comprising breed-specific data, disease predispositions, symptom identification, previous health records, and/or environmental factors, into the facial recognition algorithms.

In other preferred embodiments, these methods are included in applications for a computer, such as the computer components in a mobile phone or other computing device. These methods may be performed by cameras and computer code, the computer code resident in memory on the computing devices and being capable of being executed by one or more processors of the computing devices.

The computer executable code may be organized in terms of one or more separate or combined modules performing particular operations and/or functions or subfunctions, including (a) a scanning module for collecting and processing video imaging data, (b) a LiDAR module for collecting and processing LiDAR data, (c) an interpolating module for interpolating the video imaging data using AI and 3D Gaussian Splatting to generate a model of an animal's face, (d) an assimilating module to assimilate the 3D Gaussian Splatting model of the animal's face with the LiDAR data to generate a simulated 3D model of the animal's face, (e) a comparison module for comparing a simulated 3D model of the animal's face with a database of previously provided identification and clinical information of animals, (f) an outputting module for outputting the located identification and clinical information applicable to the animal for the use by the user, and (g) other modules providing desired functionality.

In some embodiments, at least some of these modules and/or functions or subfunctions are provided by the imaging device being used (e.g., smart phone (e.g., iphone), tablet (e.g., ipad)). In at least some of the modules, and in certain preferred embodiments, the operations and/or functions or subfunctions are provided with the application of AI (preferably provided in multiple AI modules), which is trained on known data, to improve the capability and usefulness of the modules. Thus, in certain of the most preferred embodiments of this invention, a layered ensemble of AI is used, with multiple embeddings and/or modules for machine learning tasks.

In particular, a clinical context module as described herein can serve functions such as providing clinically relevant contextual data comprising breed-specific data, disease predispositions, symptom identification, previous health records, and/or environmental factors. This may also include the use of AI (preferably provided in an associated AI module) that utilizes information from the animal medical records, geographic information, and other metadata available to the AI on the device being used and input into the patient record by the owner and/or veterinarian or other user.

Particular disease predispositions, symptom identification, and/or health diagnostics can be tailored to the species and breed of the patient being examined and/or the patient's history. Particularly relevant health issues with patients using the methods of this invention include ocular diseases and skin conditions and other medical abnormalities, where the embodiments of this invention compare the images taken and the model generated with known image and model database information and/or the patient's history, enhanced by AI to assist in the comparison and/or the detection of a medical abnormality. This can greatly speed and otherwise enhance the detection and alerting of owners and veterinary care providers of possible problems with their animals.

In certain particularly preferred embodiments, diseases and other medical abnormalities are identified using a combination of image recognition AI and clinical context AI (using a Clinical Context Module AI) on the AI generated hologram of the face of the animal that is described herein. In these embodiments, ocular disease (of the eye) and dermatologic disease (of the skin) are a focus of these methods. Other diseases, injuries and concerns that can be detected, diagnosed, tracked, and monitored include allergic responses, facial inflammation, facial trauma, dermatologic disease, neurologic disease, aural hematoma, equine encephalitis, depth of anesthesia, mucus membrane characterization, caruncle characterization, among others.

In these embodiments, some of the other diseases that can be detected (and, in some of these embodiments, monitored for change in improvement or worsening over time) can also include some or all of the following (among others not listed):
for cats and dogs; ocular disease (ocular melanoma (ocular melanoma, miosis, mydriasis, conjunctivitis, ocular discharge, trauma/infection allergy, trauma, cataracts, glaucoma, cherry eye), stress, facial alopecia (endocrine problems), tartar, gingivitis, bulging eyes, otitis externa, tumors/masses, nasal discharge, behavioral status, pain indicators, vitiligo, ring worm, mange, feline herpesvirus, jaundice, dog distemper, autoimmune dealing with nose (lupus, pemphigus), and others;
for cattle: pink eye;
for farm animals: flystrike;
for foot and mouth disease: blisters on the mouth;
for bird disease: infectious coryza, fowl pox, Newcastle disease;
for deer: chronic wasting disease; and
for horse: nasal discharge.

A display used by the user of the methods may also be included to display identification and clinical information related to the animal of interest and other information relating to the methods used by the embodiments of this invention for the benefit of the user (e.g., veterinarian).

In particularly preferred embodiments, handheld computing devices of this invention provide animal identification and clinically-relevant information. The handheld computing devices comprise (a) computer executable code that is executed by a computer processor in the handheld computing device; (b) a video camera that generates 2D video imaging data and LiDAR data relating to an animals'face; (c) a user display; and (d) wherein the computer executable code when executed (i) collects the 2D video imaging data and LiDAR data of the animal's face that originated from the video camera; (ii) interpolates the 2D video imaging data with artificial intelligence using 3D Gaussian Splatting to create a model of the animal's face; (iii) assimilates the model of the animal's face with the LiDAR data to create a simulated 3D model of the animal's face; (iv) performs a comparison of the simulated 3D model of the animal's face with a database of identification and clinical information of animals to locate any identification and clinical information applicable to the animal; and (e) outputs any located identification and clinical information for the animal to the user display.

In certain of these embodiments, the (iv) comparison comprises the application of facial recognition algorithms. In some of these embodiments, the (iv) comparison further comprises incorporating contextual data comprising breed-specific data, disease predispositions, symptom identification, previous health records, and/or environmental factors, into the facial recognition algorithms.

These methods and apparatus can be operated with an application made available to mobile phones and similar devices with computing capabilities. These methods and apparatus can also be used in managing the healthcare and other aspects of an animal's life.

Embodiments of this invention may be used by livestock managers, pet owners, veterinarians and other animal healthcare and welfare workers (e.g., dog catchers), veterinarian hospitals, municipalities and animal shelters addressing lost or missing pets and other animals, dairy or milking installations (e.g., to identify a particular animal and its feed needs, production records, progeny (e.g., calves), etc.), park rangers, zoo keepers, wildlife managers, and with wildlife conservation (e.g., with game cameras, tracking individual animals), regulatory agents and governmental agencies, among others.

As an example, veterinary hospitals can use certain embodiments of this invention for the individual and/or automatic identification of patients and other clinical applications. In some of these embodiments, the veterinary hospital can be integrated with PIMS (production or process information management systems) and/or third party analyzers to perform such identifications and provide other uses in a clinical context. In certain embodiments of this invention, the information concerning the identification and/or health diagnostics of an animal can be effectively instantly transferred into a facilities analyzers.

In certain preferred embodiments, the image information generated with this invention can be used to inform other medical devices, some of which use AI, and some that do not, with the identification of the patients, and their current image information, as well as current and previous medical information. The information generated with this invention can be used by AI robots and AI controlled functions to identify and otherwise process and treat a patient. These embodiments can be used in a surgical/pre-surgical settings by veterinary personnel, veterinary AI, and veterinary AI androids.

Certain of these embodiments can be applied to provide a registry for pets and other animals (e.g., livestock), that can be then used to identify lost animals, or provide information as to an animal's origin. With these embodiments, local, national, and international registries (e.g., subscription, free, government or municipality run) can be created of identification and other clinical information and applications. This can include the offering of rewards for lost animals and the awarding of the rewards for found animals, in certain embodiments.

In one preferred embodiment of this invention, methods for a user to return a lost animal are provided. The method comprises: (a) collecting video imaging data and LiDAR data of the lost animal's face using a camera; (b) interpolating the video imaging data with artificial intelligence using 3D Gaussian Splatting to generate a model of the lost animal's face; (c) assimilating the model of the lost animal's face with the LiDAR data to generate a simulated 3D model of the lost animal's face; (d) comparing the simulated 3D model of the lost animal's face with a database of identification of animals to locate any identification information applicable to the lost animal (e.g., by accessing a website and uploading the information to the website, which performs the comparison); (e) outputting the located identification information applicable to the lost animal to the user; and if previously provided for, (f) providing the user with any reward offered for the return of the lost animal.

Advantages of the embodiments of this invention are described and apparent throughout this specification. For example, certain embodiments of this invention have several advantages over the current methods that are used to identify animals and any clinical issues they may be expressing. These less advantageous methods include microchipping, collars, and ear tagging that fail to provide a simple, highly portable, less intrusive, less stressful, less danger-causing (e.g., infection, strangulation), automatic, and faster method and application. Further advantages will be apparent to a person of skill in the art applying embodiments of the invention.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE DRAWINGS

FIG. 1 is an example of a 3D scan of an animal face that can be used by certain embodiments of this invention.

FIG. 2 is an example of an image of an animal face that can be used by certain embodiments of this invention.

FIG. 3 is an example of an image of an animal face that can be used by certain embodiments of this invention.

FIG. 4 is an example of an image of an animal face that can be used by certain embodiments of this invention.

FIG. 5 is an example of an image of an animal face that can be used by certain embodiments of this invention.

FIG. 6 is an example of an image of an animal face that can be used by certain embodiments of this invention.

FIG. 7 is an example of a flowchart of sub-processes of a preferred embodiment of this invention.

### DETAILED DESCRIPTION OF THE INVENTION

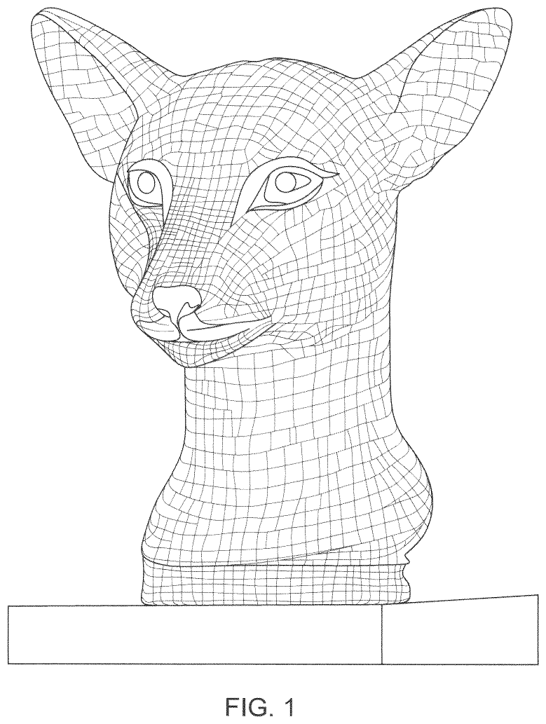

This invention comprises methods and applications for handheld and other devices (e.g., permanent installations) for identification of animals and relevant clinical information, including possible symptoms of disease, using 3D hologram generation from facial scanning and, in some embodiments, LiDAR data. These embodiments are provided for veterinary, animal welfare, livestock and other related applications that identify particular animals and possible clinical information without using invasive techniques and which facilitate early identification of disease and/or injury conditions and needed interventions, as well as providing improved animal-related tracking and record keeping. In using the terms like “facial” and “face”, this specification is using broad definitions of the terms, and they can include frontal and partial side views of the head and neck areas and not just the regions of the head around the eyes and mouth (e.g., see the region identified in FIG. 1 as an example).

Faster and improved methods of identification and/or diagnosis and uses in other clinical contexts of animals are needed. Certain embodiments of this invention provide AI-enhanced animal 3D hologram facial scanning applied to veterinary medicine. These embodiments provide methods and applications for identifying animals and retrieving their medical records swiftly and accurately. These embodiments use facial recognition algorithms to 3D scan an animal's face and immediately match it with existing profiles in a database. This capability not only streamlines the check-in process at animal hospitals but also enhances diagnostic accuracy by ensuring that veterinarians (and others) have instant access to an animal's complete medical history, previous diagnostic results, and ongoing treatment plans. This reduces the risk of medical and other errors and allows for a more personalized approach to treatment. Moreover, in emergency situations where rapid identification is crucial, these embodiments can save valuable time, facilitating immediate and effective medical intervention. These embodiments are also conducive to automation, and represent a significant step forward in veterinary diagnostics, improving efficiency and the quality of care provided to animals.

In certain embodiments of this invention, AI-enhanced animal 3D hologram facial scanning is integrated with a clinical context module in veterinary medicine and/or livestock management to significantly improve and otherwise advance disease screening and early detection. These embodiments enhance traditional facial scanning by incorporating a vast array of contextual data, such as breed-specific disease predispositions, previous health records, and environmental and other factors (e.g., lighting differences, seasonal coat differences, eye health, focus on particular features (e.g., snout condition)), into the analysis process. By doing so, these embodiments enable veterinarians and livestock managers to not only recognize physical symptoms through 3D holograms and other imaging but also to assess these symptoms in light of the animal's unique health context. This holistic approach allows for the early identification of conditions that may not be immediately apparent through physical examination alone. Consequently, these embodiments facilitate early intervention, which can dramatically improve treatment outcomes and animal/herd welfare.

Certain of the most preferred embodiments of this invention use layered ensemble AI. For example, AI can be used to standardize the 3D hologram dataset so that it is comparable to (e.g., oriented with) what is stored in, or generated from, a comparator dataset of known data (e.g., the known identity of an animal), in a manner similar to that used in facial recognition AI and software. It can also be used to focus on particular areas of animal faces for particular animal species (e.g., eyes, snouts), exclude artifacts (e.g., hands holding the animal, dirt, extra coat in the winter, cages, halters), identify differences with earlier image data of the same animal, and identify known symptoms and breed-related issues (e.g., by training the AI on adjudicated images), among other clinical uses that will be apparent to a person skilled in the art reading this specification.

Certain of the most preferred embodiments of this invention use thousands of images that are interpolated by AI (e.g., using 3D Gaussian Splatting), which create a photorealistic high resolution 3D model of the animal's face with texture mapping automatically applied by the AI. The AI in certain embodiments also utilizes LiDAR in the camera and assimilates both the LiDAR and 3D textured model created by the AI through Gaussian Splatting with thousands of images pulled from high resolution video and LiDAR scanning of the animal face by use in an iPhone (or other smart phone device) front facing camera array. Certain of these embodiments also utilize a clinical context module AI that utilizes information from the animal medical records, geographic information, and other metadata available to the AI on the mobile device and input into the patient record by the owner and/or veterinarian. The main purpose for these embodiments is to serve as a cloud based repository that links any animal medical record to the positive facial identification by any user but specifically a pet owner, government official, or a veterinary medical staff, as examples.

Certain of the most preferred embodiments house the patient's medical information and also uses a simulated 3D model of the animal that utilizes the real LiDAR 3D topographic scanning in addition to the Gaussian Splatting to increase accuracy.

These embodiments also use a clinical context module that will read the medical records and all other owner information, pet information, animal hospital information, and metadata to contribute to feed into the AI to allow for a significant increase in positive identification for veterinary medical purposes. These embodiments are also integrated on analyzers and other AI systems (e.g., as in a humanoid robot surgeon) to help identify the animal and retrieve all patient information, owner information, and other relevant data for the AI to make informed decisions by what animal and potential clinical situation it is dealing with.

Certain of the most preferred embodiments use ensemble AI modules with the clinical context module AI and the combination of LiDAR and 3D Gaussian Splatting on an iPhone (or other handheld device) to inform other AI modules, in addition to being used to make a positive identification of the animal. Thus, in these embodiments, the identification of the animal is only a small part of what these embodiments can accomplish. An important purpose of certain of these embodiments is to apply the data to AI systems only one of which is used to identify the animal.

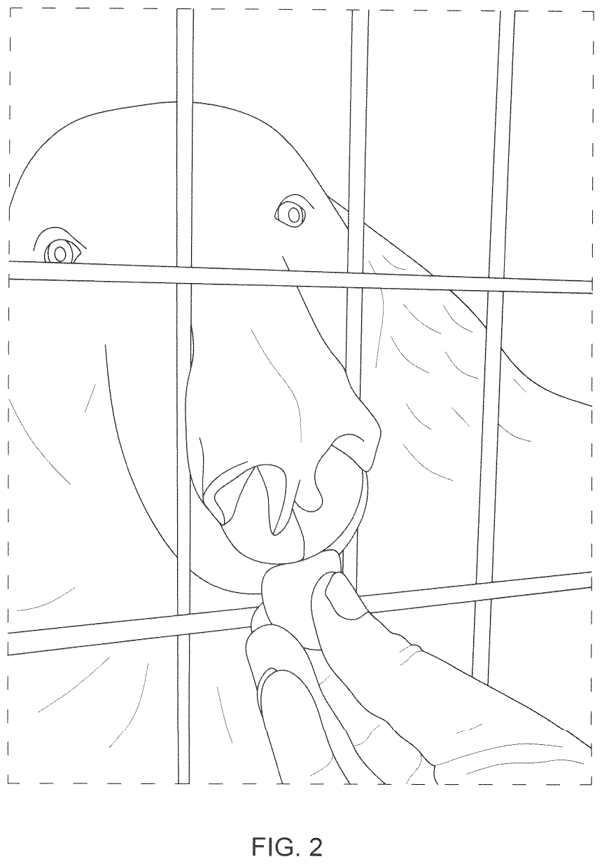

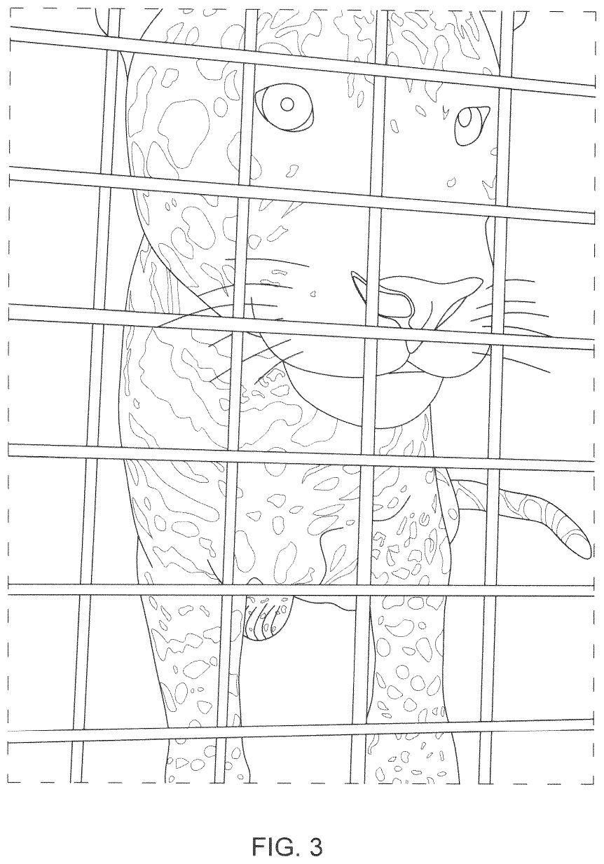

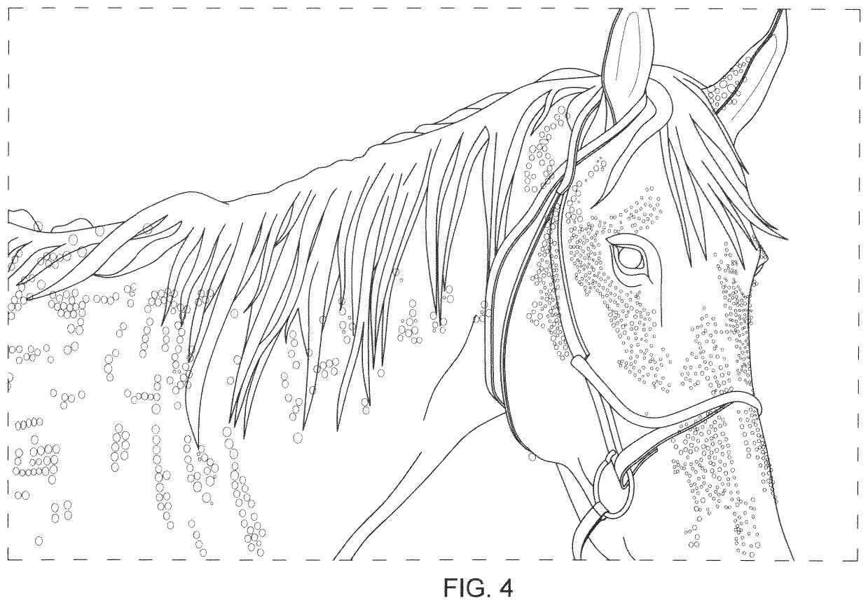

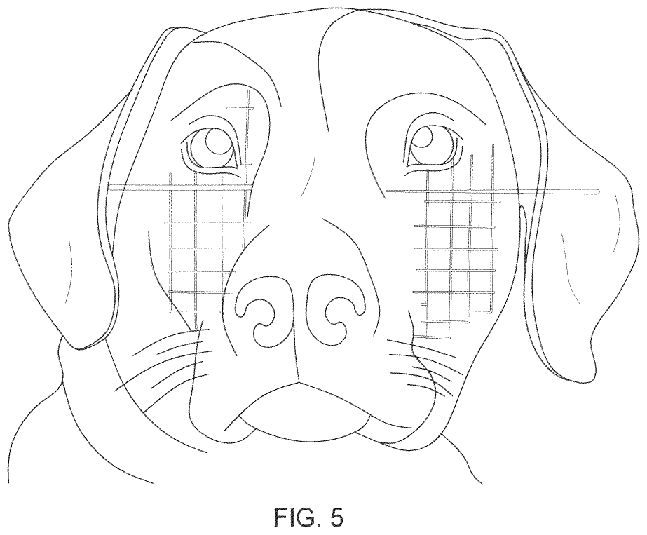

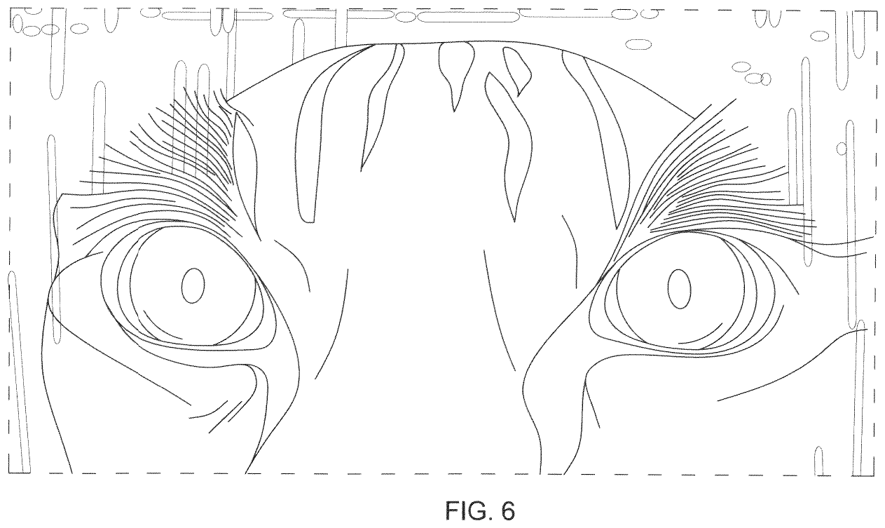

FIG. 1 shows an exemplary schematic of a facial scan that can be used in embodiments of this invention. FIGS. 2-6 show schematics of image examples of different animals that can be used in embodiments of this invention. The AI used herein can be applied in these latter figures to orient the scans of these partial side views and to identify fiducial points for comparison and effectively ignore enclosure and other artifacts (e.g., cages, halters) to provide more useful data sets of particular animals and their situations.

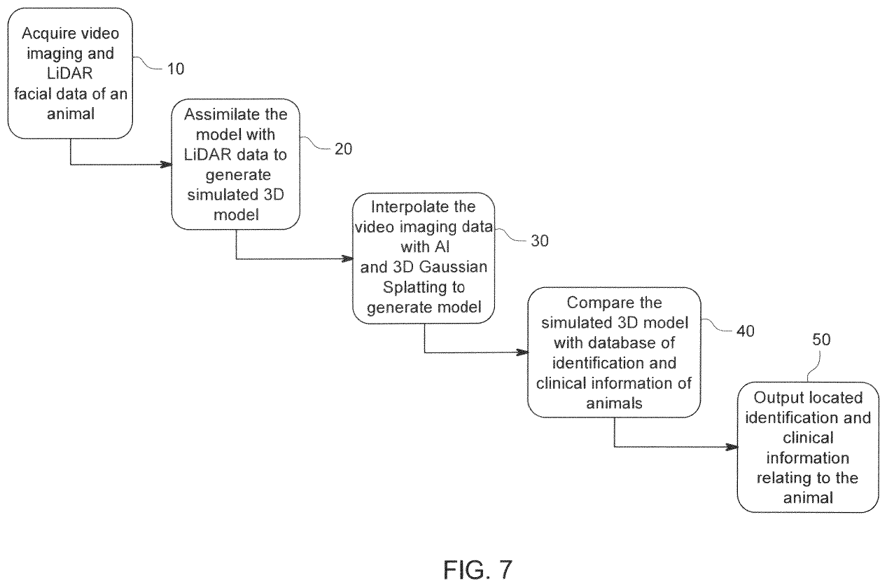

FIG. 7 shows an embodiment of a flowchart of subprocesses (or functions or operations) of a preferred method of this invention. This flowchart includes (a) acquire (e.g., collect from a video camera and/or memory) video imaging and LiDAR facial data of an animal 10, (b) interpolate the video imaging data with AI and 3D Gaussian Splatting to generate a model (e.g., a first model based on the video imaging data) 20, (c) assimilate the model with LiDAR data to generate a simulated 3D model (e.g., a second model based on the first model assimilated with the LiDAR data) 30, (d) compare the simulated 3D model with a database of identification and clinical information of animals 40 (e.g., a local, regional, national, or international registry; a database stored on a local or cloud-based server) to locate identification and clinical information relating to the animal, and (e) output located identification and clinical information relating to the animal 50. Each of these subprocesses or steps can be enhanced by using AI (e.g., in the form of modules) to increase the speed and/or accuracy of the operation being performed.

The subject matter of this disclosure is now described with reference to the following examples. These examples are provided for the purpose of illustration only, and the subject matter is not limited to these examples, but rather encompasses all variations which are evident as a result of the teaching provided herein.

### Example 1

An animal is in the sitting or standing position in front of the pet owner. A video of the animal's face is scanned by a smart phone device. The animal's facial scan video is interpolated using AI to create a detailed and unique 3D topographic map of the 3D facial structures and fine 3D detail of the nose topography to create a completely unique identifying 3D facial signature for that individual animal. The animal's 3D facial scan is used to attach identifying information of the animal including name, age, reproductive status, vaccination history, detailed up-to-date medical records, owner information, owner address, and owner phone number. This identifying 3D facial signature is also usable to populate demographic and identifying information on other medical devices and software applications automatically. This information can be stored in cloud-based servers accessible broadly.

As facial features change throughout the animal's life, an updated scan may be required to continue to positively identify the individual animal as they grow and experience facial structural or topographic change. In some embodiments, AI is used to predict and account for such changes.

Upon completion of initial 3D facial scanning, the animal information will be submitted into an international registry of all species and be retrievable upon positive 3D facial identification of the individual animal by any individual with a smart phone device. AI will be utilized in the aid of positive identification of the individual animal in the event of debris or mud partially obscuring the topographic features of the animal attempting to be identified, low or high level lighting partially obscuring the topographic features of the animal attempting to be identified, tissue scars and other natural changes to topographic facial features partially obscuring the topographic features of the animal attempting to be identified, or condensation or water partially obscuring the topographic features of the animal attempting to be identified, and cages bars or glass barriers that may interfere partially obscuring the topographic features of the animal attempting to be identified, or condensation or water partially obscuring the topographic features of the animal attempting to be identified. This identification method will be utilized in robotic doctors and or surgeons, drones, and artificial intelligence systems for the automatic identification of individual animals by such systems in the applications of lost or stray animals, meat and production animals, zoo and wildlife animals and endangered or de-extinct species, and animal hospital veterinary medical functions, among others.

In some embodiments, the LiDAR data from the camera in the smart phone device is also used by itself or interpolated with the facial scan data that is collected.

### Example 2

A veterinary office includes a mobile phone with an application that practices embodiments of this invention. The veterinarian or other animal healthcare worker in relation to a patient visit (e.g., examination) opens the application in the mobile phone and uses the camera feature to take a video scan of the face of a patient (e.g., dog, cat, or other animal). The application creates a 3D hologram dataset from the scan and compares the hologram dataset to the veterinarian office's database. In some embodiments, the LiDAR data from the camera is also used. If a match occurs, the patient's identification and medical information (e.g., history, reason for visit) is presented on the mobile phone screen and/or another accessible screen for the veterinarian and/or other animal healthcare worker to see. Additional medical information for that patient can also be saved to the veterinarian office's database during or after the patient visit. The method may also compare previous images taken of the patient, and provide analyses of symptoms of disease, injuries and other changes or abnormalities that may be expressed on an animal's face.

The application in certain embodiments can also access a local, national, and/or international registry/database with respect to the patient and save or access identification and medical information there.

### Example 3

An animal shelter includes a mobile phone with an application that practices embodiments of this invention. An animal shelter worker opens the application in the mobile phone and uses the camera feature to take a video scan of the face of a lost animal (e.g., dog, cat, or other animal). The application creates a 3D hologram image dataset from the scan and compares the dataset to a local, national, and/or international registry/dataset to attempt to identify the lost animal and return it to its owner. In some embodiments, the LiDAR data from the camera is also used.

In certain embodiments, a local municipality requires or accepts voluntarily provided images of the face of local pets with respect to pet licensing and/or as desired by residents. The images are used to create a database of the local pets that can be accessed by veterinarians, animal shelters, animal welfare workers, dog catchers, and other interested parties to help return and otherwise manage local pets.

In these embodiments, the method may also compare previous images taken of the animal, and provide analyses of symptoms of disease, injuries and other changes or abnormalities that may be expressed on an animal's face.

### Example 4

A dairy or cow milking facility includes a mobile device (e.g., tablet, mobile phone) with an application that practices embodiments of this invention. A facility worker opens the application in the mobile device and uses the camera feature to take a video scan of the face of a cow. The application creates a 3D hologram dataset from the scan and compares the hologram dataset to the facility's database. In some embodiments, the LiDAR data from the camera is also used. If a match occurs, the cow's identification and other information (e.g., history, feeding requirements, calving situation) is presented on the mobile device and/or another accessible screen for the facility worker to see. Additional information for that cow can also be saved to the facility's database (e.g., feeding, weight after stepping on scale) after a particular use.

The method may also compare previous images taken of the cow, and provide analyses of symptoms of disease, injuries and other changes or abnormalities that may be expressed on a cow's face.

The application in certain embodiments can also access a local, national, and/or international registry/database with respect to the cow and save or access identification and other information there. Governmental authorities can use the information to correlate the cow's information with particular products (e.g., milk, meat) and enable tracing in case of food borne illnesses and other issues.

### Example 5

A livestock slaughtering facility includes a permanent device (e.g., mounted camera) with an application program that practices embodiments of this invention. The device is located in a particular area where an animal passes (e.g., a single-file cattle shoot with a scale) and it automatically takes images of the animal's face when it passes. The application creates a 3D hologram dataset from the scan and stores it along with other information concerning the animal (e.g., weight, time and date of recording, identification information). In some embodiments, the LiDAR data from the camera is also used. In some embodiments, the application compares the 3D hologram dataset and/or the LiDAR data to the information in a database to identify the animal and/or provide additional information concerning the animal (e.g., identify any medical abnormalities).

The application in certain embodiments can also access a local, national, and/or international registry/database with respect to the animal and save or access identification and other information there. Governmental authorities can use the information to correlate the animal's information with particular products (e.g., meat, hide) and enable tracing in case of food borne illnesses and other issues.

### Example 6

An animal identification process of this invention is practiced wherein an animal owner puts out an alert that an animal is missing and offers a reward for its location and/or return, a person finds the animal and scans its face and then uses the process to identify the animal and alert the owner of the animal's location and/or return, and the reward is automatically provided to the person who finds the animal through the process.

In certain of these embodiments, the animal owner is capable of loading pre-authorized emergency vet funds into an account that effectively provides for the animal to pay for vet care without the owner being present in the event the animal is lost or presents to the emergency room without the owner.

### Particular Applications to Computer Devices

The system applied to this invention may include a plurality of different computing device types. In general, a computing device type may be a computer system or computer server. The computing device may be described in the general context of computer system executable instructions, such as program modules, being executed by a computer system (described for example, below). In some embodiments, the computing device may be a cloud computing node (for example, in the role of a computer server) connected to a cloud computing network (not shown). The computing device may be practiced in distributed cloud computing environments where tasks are performed by remote processing devices that are linked through a communications network. In a distributed cloud computing environment, program modules may be located in both local and remote computer system storage media including memory storage devices.

The computing device may typically include a variety of computer system readable media. Such media could be chosen from any available media that is accessible by the computing device, including non-transitory, volatile and non-volatile media, removable and non-removable media. The system memory could include random access memory (RAM) and/or a cache memory. A storage system can be provided for reading from and writing to a non-removable, non-volatile magnetic media device. The system memory may include at least one program product having a set (e.g., at least one) of program modules that are configured to carry out the functions of embodiments of the invention. The program product/utility, having a set (at least one) of program modules, may be stored in the system memory. The program modules generally carry out the functions and/or methodologies of embodiments of the invention as described herein.

As will be appreciated by one skilled in the art, aspects of the disclosed invention may be embodied as a system, method or process, or computer program product such as an application. Accordingly, aspects of the disclosed invention may take the form of an entirely hardware embodiment, an entirely software embodiment (including firmware, resident software, micro-code, etc.) or an embodiment combining software and hardware aspects “system.” Furthermore, aspects of the disclosed invention may take the form of a computer program product embodied in one or more computer readable media having computer readable program code embodied thereon.

Aspects of the disclosed invention are described above with reference to methods, apparatus (systems) and computer program products according to embodiments of the invention. It will be understood that each method can be implemented by computer program instructions. These computer program instructions may be provided to the processor of a general-purpose computer, special purpose computer, or other programmable data processing apparatus to produce a machine, such that the instructions, which execute via the processor of the computer or other programmable data processing apparatus, create means for implementing the functions/acts specified in a flowchart and/or block diagram block or blocks.

### Other Embodiments

Although the present invention has been described with reference to teaching, examples and preferred embodiments, one skilled in the art can easily ascertain its essential characteristics, and without departing from the spirit and scope thereof can make various changes and modifications of the invention to adapt it to various usages and conditions. Those skilled in the art will recognize or be able to ascertain using no more than routine experimentation, many equivalents to the specific embodiments of the invention described herein. Such equivalents are encompassed by the scope of the present invention.

What is claimed is:

1. A method for identification of an animal and providing clinically-relevant information concerning the animal to a user, the method comprising:
(a) collecting video imaging data and LiDAR data of the animal's face using a camera;
(b) interpolating the video imaging data with artificial intelligence using 3D Gaussian Splatting to generate a model of the animal's face;
(c) assimilating the model of the animal's face with the LiDAR data to generate a simulated 3D model of the animal's face;
(d) comparing the simulated 3D model of the animal's face with a database of identification and clinical information of animals to locate any identification and clinical information applicable to the animal; and
(e) outputting the located identification and clinical information applicable to the animal to the user.

2. The method of claim 1, wherein the (d) comparing comprises application of facial recognition algorithms.

3. The method of claim 2, wherein the (d) comparing further comprises incorporating contextual data comprising breed-specific data, disease predispositions, symptom identification, previous health records, and/or environmental factors, into the facial recognition algorithms.

4. The method of claim 1, wherein the (d) comparing further comprises using artificial intelligence to locate any medical abnormality information.

5. A handheld computing device that provides animal identification and clinically-relevant information, the handheld computing device comprising:
(a) computer executable code that is executed by a computer processor in the
handheld computing device;
(b) a video camera that generates 2D video imaging data and LiDAR data relating to an animal's face;
(c) a user display; and
(d) wherein the computer executable code when executed (i) collects the 2D video imaging data and LiDAR data of the animal's face generated by the video camera; (ii) interpolates the 2D video imaging data with artificial intelligence using 3D Gaussian Splatting to generate a model of the animal's face; (iii) assimilates the model of the animal's face with the LiDAR data to generate a simulated 3D model of the animal's face; (iv) performs a comparison of the simulated 3D model of the animal's face with a database of identification and clinical information of animals to locate identification and clinical information applicable to the animal; and (v) outputs the located identification and clinical information applicable to the animal to the user display.

6. The handheld computing device of claim 5, wherein the (iv) comparison comprises the application of facial recognition algorithms.

7. The handheld computing device of claim 6, wherein the (iv) comparison further comprises incorporating contextual data comprising breed-specific data, disease predispositions, symptom identification, previous health records, and/or environmental factors, into the facial recognition algorithms.

8. The handheld computing device of claim 5, wherein the (iv) comparing further comprises using artificial intelligence to locate any medical abnormality information.

9. An application for identification of an animal and providing clinical information concerning the animal for use with a mobile phone, the mobile phone having (a) a computer processer, (b) a video camera that can generate 2D video imaging data and LiDAR data relating to the face of the animal; and (c) a user display, the application comprising:
(a) computer executable code that is executed by the mobile phone's computer processor;
(b) wherein the computer executable code when executed (i) collects the 2D video imaging data and LiDAR data of the animal's face that is generated by the video camera; (ii) interpolates the 2D video imaging data with artificial intelligence using 3D Gaussian Splatting to create a model of the animal's face; (iii) assimilates the model of the animal's face with the LiDAR data to create a simulated 3D model of the animal's face; (iv) performs a comparison of the simulated 3D model of the animal's face with a database of identification and clinical information of animals to locate identification and clinical information applicable to the animal; and (v) outputs the located identification and clinical information applicable to the animal to the user display.

10. The application of claim 9, wherein the (iv) comparison comprises the application of facial recognition algorithms.

11. The application of claim 10, wherein the (iv) comparison further comprises incorporating contextual data comprising breed-specific data, disease predispositions, symptom identification, previous health records, and/or environmental factors, into the facial recognition algorithms.

12. The application of claim 9, wherein the (iv) comparing further comprises using artificial intelligence to locate any medical abnormality information.

13. A method for managing the healthcare of an animal, the method comprising
(a) identifying the animal and clinical information regarding the animal by:
(i) collecting video imaging data and LiDAR data of the animal's face using a camera;
(ii) interpolating the video imaging data with artificial intelligence using 3D Gaussian Splatting to generate a model of the animal's face;
(iii) assimilating the model of the animal's face with the LiDAR data to generate a simulated 3D model of the animal's face;
(iv) performing a comparison of the simulated 3D model of the animal's face with one or more databases of identification and clinical information of animals to locate any identification and clinical information applicable to the animal; and
(v) outputting the located identification and clinical information applicable to the animal to the user;
(b) updating the identification and clinical information applicable to the animal in the one or more databases with any changes to such; and
(c) maintaining the updated identification and clinical information applicable to the animal in the one or more databases.

14. The method of claim 13, wherein the (iv) comparison comprises the application of facial recognition algorithms.

15. The method of claim 14, wherein the (iv) comparison further comprises incorporating contextual data comprising breed-specific data, disease predispositions, symptom identification, previous health records, and/or environmental factors, into the facial recognition algorithms.

16. The method of claim 13, wherein the (iv) comparing further comprises using artificial intelligence to locate any medical abnormality information.

17. A method for a user to return a lost animal, the method comprising
(a) collecting video imaging data and LiDAR data of the lost animal's face using a camera;
(b) interpolating the video imaging data with artificial intelligence using 3D Gaussian Splatting to generate a model of the lost animal's face;
(c) assimilating the model of the lost animal's face with the LiDAR data to generate a simulated 3D model of the lost animal's face;
(d) comparing the simulated 3D model of the lost animal's face with a database of identification of animals to locate any identification information applicable to the animal;
(e) outputting the located identification information applicable to the lost animal to the user; and
(f) providing the user with any reward offered for the return of the lost animal.

18. The method of claim 17, wherein the (d) comparison comprises the application of facial recognition algorithms.

19. The method of claim 18, wherein the (d) comparison further comprises incorporating contextual data comprising breed-specific data, disease predispositions, symptom identification, previous health records, and/or environmental factors, into the facial recognition algorithms.

20. The method of claim 17, wherein the (d) comparing further comprises using artificial intelligence to locate any medical abnormality information.
